(19) United States
(12) Patent Application Publication
(10) Pub. No.: US 2004/0024285 A1
(43) Pub. Date: Feb. 5, 2004

(54) Blood pump with impeller

(76) Inventor: Helmut Muckter, Aachen (DE)

Correspondence Address: Ursula B. Day, Suite 4714, 350 Fifth Avenue, New York, NY, 10118, US

(21) Appl. No.: 10/601,280

(22) Filed: Jun. 20, 2003

Related U.S. Application Data
(60) Provisional application No. 60/390,416, filed on Jun. 21, 2002.

(30) Foreign Application Priority Data
Jun. 21, 2002 (DE) DE 102 27 886.5
Jul. 12, 2002 (DE) DE 102 31 479.9

(51) Int. Cl. A61M 1/12

(52) U.S. Cl. 600/16; 623/3.13; 623/3.26

(57) ABSTRACT

A blood pump having a pump housing and an impeller disposed in the housing and driven by an electric drive includes at least two connection devices directly disposed at the housing for connection to an artery outside the heart such as for example a suture ring or a vascular prosthesis and configured in such a manner to realize a direct connection of the artery to the pump housing, and wherein a pump conduit can be implanted into the aorta ascendens for relieving the left ventricle and in the truncus pulmonalis or into the pulmonalis furcation for reliving the right cardiac ventricle and other blood vessels for improving the blood circulation or elevating the pressure in a certain vascular section.

### CROSS-REFERENCES TO RELATED APPLICATIONS

This application claims the priority of German Patent Application Serial No. DE 102 27 886.5, filed Jun. 21, 2002, pursuant to 35 U.S.C. 119(a)-(d), the subject matter of which is incorporated herein by reference.

This application claims the priority of German Patent Application Serial No. DE 102 31 479.9, filed Jul. 12, 2002, pursuantto 35 U.S.C. 119(a)-(d), the subject matter of which is incorporated herein by reference.

This application claims the benefit of prior filed provisional application, Appl. No. 60/390,416, filed Jun. 21, 2002, pursuant to 35 U.S.C. 119(e), the subject matter of which is incorporated herein by reference.

### BACKGROUND OF THE INVENTION

The present invention relates to a blood pump, and in particular to a blood pump having an impeller.

Within the medical field of artificial organs or implants, a variety of pumps are now known and utilized. These pumps are for example, utilized as mechanical implants for supporting or supplementing cardiac functions, or they may be utilized to take over an entire cardiac function such as for example replacing the function of one or both cardiac ventricles or for supporting the blood flow in a blood vessel.

Part of the prior art are, on the one hand, membrane pumps provided with integrated mitral valves that are operated by mechanical, hydraulically or pneumatic means. On the other hand, various types of impeller pumps are utilized that include a radial, semi-axial, or respectively a diagonal or axial impeller, as are for example described in WO/97/49439 and in U.S. Pat. No. 4, 994, 078. Pumps of this type are implanted with the aid of various techniques for blood circulatory connections. The following are three basic situations where blood pumps are predominantly applied:

1. Replacement of the heart: A complete replacement where of the heart is replacedwith a new pump system. The damaged heart is entirely removed and replaced by a mechanical artificial heart.

2. Bypass systems: In this system, the damaged heart remains in situ and the circulatory blood is guided via tubes through an intra- or extra-corporial pumping system by by-passing either entirely or partially one or two of the heart ventricles.

3. Intravascular impeller systems: In this technique, a small impeller pump which is integrated in a tube is led via the aorta retrograde through the aortic valve into the left ventricle. The circulatory blood is guided by means of the impeller pump from the ventricle into the aorta.

Depending on the type of pump and the various techniques wherein they are utilized in the medical field, a variety of problems are known to exist.

For example, there are the thrombo-embolic complications. In this situation, a rather large amount of surface of foreign bodies is associated with most of the systems utilized, especially when there is the need for using extensive tubing, there is always the threat of blood clot formation even when the highest permissible doses of blood coagulation inhibitors haven been administered.

Further complications are arising in bleeding situations, for example where the administration of large doses of blood coagulation inhibitors, which may have become necessary to prevent thrombo-embolic complications, can lead to a severe blood coagulation diminishment, which can then lead to life-threatening hemorrhaging.

In situations where there is insufficient auxiliary safety support during system failure, so that even only a short break in the system can lead to acute life threatening conditions, as for example is known in pump systems of the prior art.

Threat of infection poses another risk due to the rather large plastic surfaces presented by tubing, which provides a breading ground for bacteria.

Furthermore, impairment of the heart function can occur through the pump system itself. Most pump systems known in the prior art are also known to have a negative impact on the heart function, which is to a great extent based on their design. This is due in part to the large volume tubes, which uptake blood from the vestibule or the tip of the ventricle and there impair the blood flow or the function of the heart valves. Moreover, the tubes, which are pushed through the heart valves can impair the heart valve function and to thereby obstruct the flow diameter of the heart valve.

It would therefore be desirable and advantageous to provide an improved blood pump to obviate drawbacks of the prior art and to provide a blood pump which is insertable into a portion of a blood vessel or artery without the use of tubes.

### SUMMARY OF THE INVENTION

According to one aspect of the present invention, a blood pump with an impeller is provided which can be utilized in a vascular section without the use of tubes.

According to another aspect of the invention, the blood pump having an impeller includes at least two vascular connections for joining the pump to a blood vessel outside the heart such as for example used with a suture ring or vascular prosthesis. With a system of these vascular connections, the impeller pump can be connected directly with the blood vessel and without the use of tubes. With this arrangement, it is possible to use the blood pump for example between two vascular ends, for example after a section in a portion of the aorta or in the area of a vascular furcation.

Another feature of the pump system according to the present invention is that it can replace the entire function or only a partial function of one or both heart ventricles. In addition, with the pump system according to the present invention, an effective pressure release in one or both ventricles can be attained so that a reduction of a dilated heart cavity or cavities is effectively realized. Since the pump according to the present invention can be arranged outside the heart, the blood flow through the heart cavity can be maintained during the pumping support, so that an intra-cavity stasis can be avoided and to thereby counteract any clot formation in the heart cavities. With the pump configured according to the present invention, the pump system can even be utilized where an implanted mechanical heart valve prostheses is in an aortic position or a pulmonary position.

A further feature of the present invention is that due to the direct vascular connection of the connection system of the blood pump and the lack of any unnecessary tubes, the size of the pump can be kept small thereby leading to the desired minimal amount of body foreign surfaces near blood and vascular tissue. As a result, the risk of clot formation as well as the risk of any bacterial growth can be kept to a minimum. Furthermore, the system is cost effective by keeping the surfaces of body foreign objects comprised of highly biocompatible and very expensive materials coming in contact with blood and tissue to a minimum.

A further feature of the present invention is its easy removal. For example, the implanted pump can be implanted in such a manner that after sufficient recovery of the heart function, it can be removed in a simple manner by simple surgical steps.

The blood pump is configured in such a way that when there is demand for supporting the left ventricle, it can be implanted in the form of a pumping conduit into the aorta ascendens, and for support of the right ventricle in the form of a pumping conduit into the truncus pulmonalis or into pulmonary furcation. Furthermore, to increase the blood flow or raising the pressure in a certain vascular section, the pump can be implanted into the corresponding in-flow blood vessel. When treating peripheral occlusive arterial disease or when carrying out dialysis or extra-corporeal membrane oxygenation (ECMO), the blood pump is implanted in the form of a pump conduit into the main artery or blood vessel which provides the respective vascular section with blood flow. When using the pump for support of one or two heart ventricles, the pump is preferably implanted in such a manner that the function of the natural cardiac valves, that is, the aortic valve, respectively the pulmonary valve, or the function of a possibly implanted cardiac valve prostheses, if that were the case, are not impaired.

Another advantageous feature according to the present invention is a disposition of the vascular connection system directly Mat the pump housing. Accordingly, the vascular connection system can include a pump housing and an impeller driven by electric motor disposed in the housing and, the pump can be provided with at least two connection devices for connecting to a blood vessel outside of the heart such as for example with a suture ring orvascular prosthesis and configured in such a manner that a direct connection of the blood vessel to the pump housing can be realized, thus leading to a compact assembly of the pump and fewer necessary parts.

A particularly compact embodiment of the pump according to the present invention is realized when the length of the pump housing is less than twice its diameter, preferably 1.5 times its diameter. Thus, when carrying out the implantation of the blood pump, only a short piece of blood vessel has to be removed, which in a sense is then replaced by the conduit.

It is s further advantageous feature of the present invention, when the length of the vascular connection system is configured with an especially short conduit in order to keep damage to a minimum and to possibly cut only a short section of the blood vessel. The vascular connection system should preferably be shorter than the diameter of the intermediary piece.

Assembly of the pump is realized in a simple manner and includes a motor and a pump housing having webs that are preferably configured as vanes that are disposed between the motor and the outer casing of the housing. The vanes are thus supporting the motor preferably in co-axial position relative to the pump housing and at the same time carry out its key function on the flow through circulatory blood.

The Vanes have preferably a stable configuration so that they can securely support the motor in the pump housing even during torsional changes.

As a power supply for the motor, it is contemplated that the required cables or metal pins are extending in the interior of the webs which are configured as vanes so that that any contact of the cables or pins with blood will be prevented.

In another advantageous configuration of the blood pump according to the present invention, as a safety feature, the system will be maintained in such a manner that during a possible system failure, other than the immediate non-available cardiac support, there will be no further negative impact on the system which might impair the circulation or the pulmonary function. This safety feature is realized by providing that the area between the motor and the outer casing of the pump housing has a free flow diameter of at least 50%, preferably 80% of the free flow diameter at one end of the intermediary piece. Upon a pump failure, blood can flow virtually unobstructed through the pump using it as a conduit, to thereby maintain the blood circulatory function.

In a special embodiment of the pump according to the invention, the pump is provided with two motors, two pump housings and one housing connection. A connection system of this type can be adapted for optimal layout of the pumps in such a manner that the least amount of outer torque is generated. This can be realized by driving the impellers in opposite direction for example by motors that are driven in opposite direction. Thus, the mass moment of inertia is preferably arranged in such a manner that under dynamic operation or respectively, pulsed operation, an as small as possible outer torsion will be generated. This can be realized when the impellers are driven in opposite direction which can be done by opposing driving motors. The mass inertia moments of the rotating parts are accordingly arranged so that during dynamic, respectively pulsatile operation, only very small torsional momentum are generated.

In another embodiment, the pump according to the present invention has an auxiliary electromechanical motor disposed in the pump housing, which motor rotationally drives a mass in opposite direction relative to the rotation of the impeller. This also permits the outer momentum to be kept small. In this particular embodiment, an additional electric drive is connected to the system driving a rotating mass in opposite direction and synchronous to the rotation of the rotor shaft of the blood pump, and the system is designed so that upon changing the torsion, the mass moment of inertia results in only an insignificant outward momentum.

In a further advantageous embodiment, the housing which houses the blood pump is provided with an attachment device, whereby the blood pump can be attached to tissue, preferably at the bones of the rib cage. This arrangement is carried out by connecting an adaptable attachment device to the housing so as to provide a connection to the surrounding tissue structures which are capable of absorbing a momentum which may be generated under either dynamic or pulsatile operation.

### BRIEF DESCRIPTION OF THE DRAWING,

Other features and advantages of the present invention will be more readily apparent upon reading the following description of currently preferred exemplified embodiments of the invention with reference to the accompanying drawing, in which:

FIG. 1 is a section of a blood pump according to the present invention implanted in the aorta ascendens;

FIG. 2 is a partial front sectional view of a blood pump with two pump housings for support of two cardiac chambers; and

FIG. 3 is a partial front sectional view of a blood pump for implantation into the furcation of the pulmonary stem.

### DETAILED DESCRIPTION OF PREFERRED EMBODIMENTS

Throughout all the Figures, same or corresponding elements are generally indicated by same reference numerals.

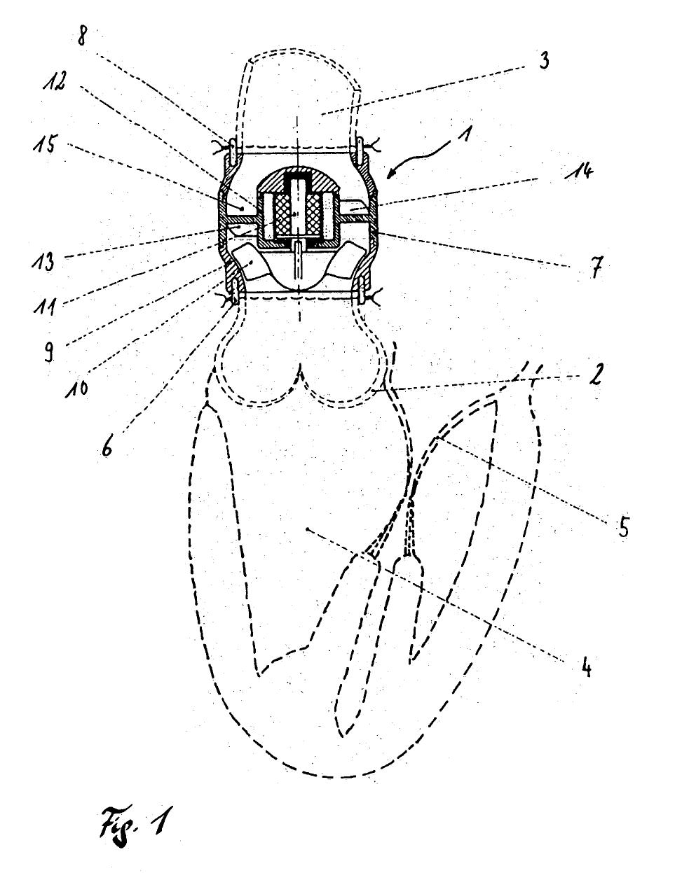

Turning now to the drawing, and in particular to FIG. 1, there is shown a blood pump 1 which is implanted between the aortic valve, 2 and the aorta ascendens 3. The cardiac areas and the aorta ascendens 3 are shown in broken lines. The cardiac structures shown are the left cardiac ventricle 4, the mitral valve 5 and the aortic valve 2.

The pump housing 7 which serves as conduit is sutured at a secure distance to the aortic valve with a suture ring 6. The end 7 situated in opposite relationship is connected by means of a suture ring 8 to the aorta ascendens. A segment of the aorta ascendens corresponding to the size of the pump conduit is trimmed away to allow implantation of the blood pump. Thus, the suture rings 6 and 8 serve as a vascular connective system when implantating the blood pump 1. The blood pump 1 consists essentially of a pump housing 7 and an impeller 10 disposed in the housing with a semi-axial, respectively a diagonal guide means, and a fully enclosed motor drive 11. The motor housing 12 is firmly connected, to the outer casing 9 of the pump housing 7 by means of webs 13, 14. These webs 13, 14 are configured as Vanes for conducting the blood flow and house the power supply for the motor drive for which cables or pins can be used

The impeller pump can be adapted to any suitable configuration for radial, semi-radial or axial flow conditions known in the field of flow mechanics to be Utilized for an impeller of corresponding design for a blood pump 1. Likewise, any type of parts that are suitable for blood pumps such as, for example, shaft support, shaft seal, motor housing seal and power transmission, in particular also magnetic power transmission for a touchless drive of the impeller, and that are suitable for, blood pumps, can be successfully utilized. It is particularly advantageous if the blood pump 1 is operated by an intelligent system. Accordingly, such a system incorporates devices for the control and regulation of the arterial blood pressure as well as the pressure in the ventricle.

In an advantageous variant of this embodiment of the blood pump 1, a pulsatile operation of the impeller pump can also be realized through the control device, whereby the pulsation of the impeller pump 1 can be adapted to the natural rhythm of the, heartbeat, which may preferably be effected for example through a pressure trigger or an EKG-trigger.

In the embodiment of the pump as, shown in FIGS. 1-3, the shape of the outer casing 9 of the pump housing 7 is configured in such a fashion that the free flow diameter 15 is substantially undiminished with respect to a hemo-dynamically relevant stenosis.

With the pump 1 according the present invention, powering down the system can easily be carried out for example when a system defect occurs or within a certain weaning phase after cardiac recovery, the impeller pump can be entirely or partially shut down without having a negative impact on the blood circulation function even if there is no longer any cardiac support or only reduced support. In the case of a possible explantation which may appear as indicated, the pump 1 can be replaced by utilization of a conventional vascular, interpose such as the utilization of a heart-lung machine.

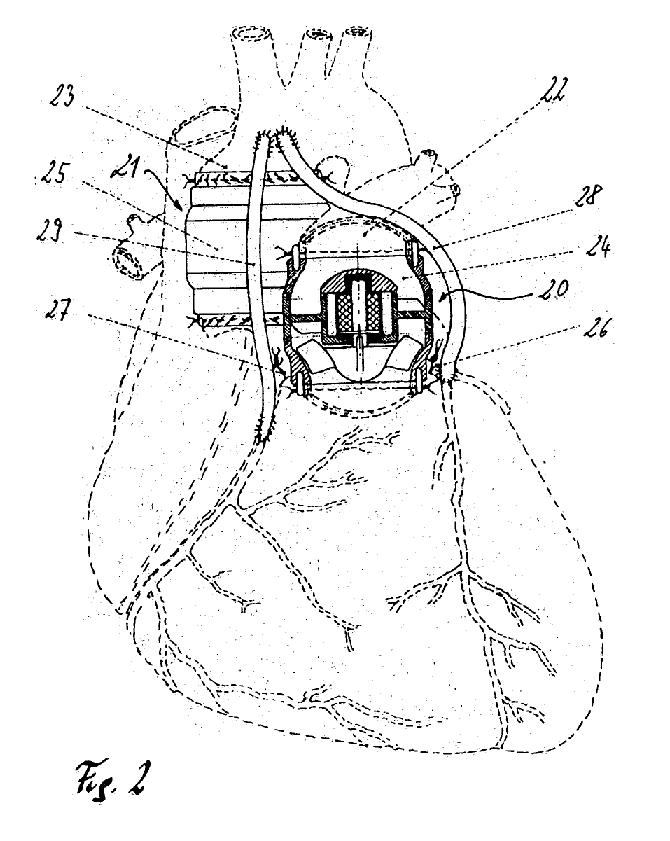

FIG. 2 shows the use of blood pump having two housings 20, 21 suitable for the support of both cardiac chambers. Of pump conduits 24, 25, on conduit each is implanted into respectively truncus pulmonalis 22 and the aorta ascendens 23 in sufficient distance from the cardiac valves to thereby realize a partial or complete support of the heart. In order to ensure a sufficient blood supply to the coronary arteries, it is advantageous to centrally close the stems of the coronary arteries in the bulbus of the aorta at points 26, 27 at the low pressure side of the pump conduit 25 and to establish venous by-passes 28 and 29 or to establish anastomosis with one or both mammary arteries to increase circulation of the coronary arteries.

Preferably, the pump conduits 24 and 25 are operated by pulsatile trigger. Furthermore, in order to avoid generating outer momentum, the pump conduits 24, 25 are firmly connected to one another by means of adjustable rod linkage not shown here in detail. The impellers are driven in opposite rotation and the rotating parts are configured to minimize the mass inertia momentum and that any torsional change in the pump dynamic results in only a negligible amount of outer momentum.

The principle of active mass equalization is likewise applicable with pumps as shown in FIG. 1 having only one housing. To create this effect, a rotating mass, not shown here, and driven by an electric motor, which is not shown here, is connected to the pump housing 7 and rotates in opposing manner synchronous to the drive motor 11 of blood pump 1 and which has a mass inertia moment such that a change in torsion results in only a very insubstantial or negligible outer momentum.

The pumps shown in FIG. 1 as well as the pump as shown in FIG. 2 can be advantageously fixed to the bony part of the rib cage by means of a suitably adapted attachment device, not shown here.

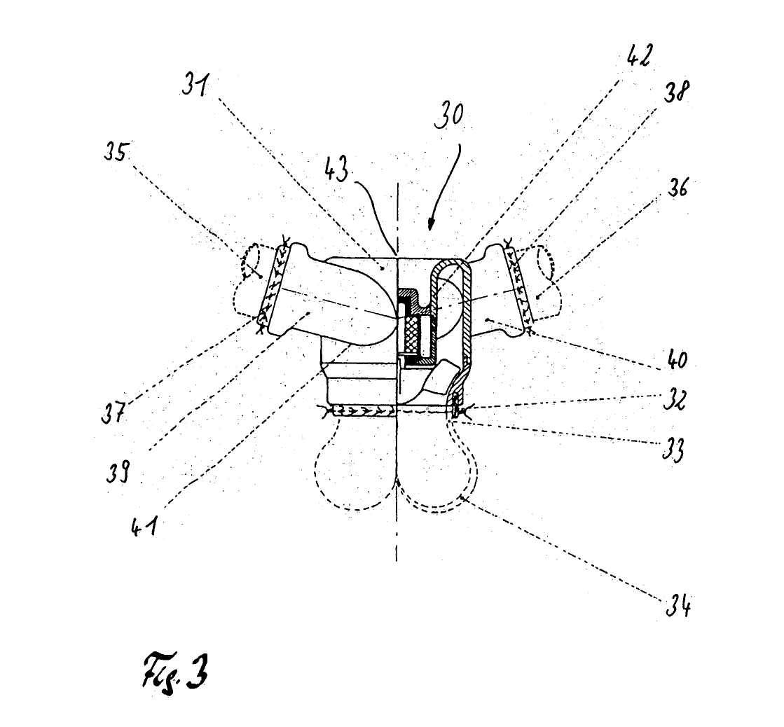

In FIG. 3 another embodiment of a blood pump 30 provided with a variant of the pump conduit 31 and is particularly suited for a very short truncus pulmonalis for support of the right cardiac ventricle. In this type of configuration, the pump conduit 31 has a Y-shape. A suture ring 32 is disposed at the inflow side, which is sutured to the stem of the pulmonary artery 33 directly behind the pulmonary valve 34. On the outflow side, the right and the left pulmonary artery 35 respectively 36 are directly connected to the out-flow connecting piece 39, 40 of the pump conduit 31 by means of suture rings 37, 38. The connecting pieces 39, 40 preferably lead, in correspondence to the rotational direction of the pump 30 in eccentric manner into the pump housing 41. The attachment of the motor drive in the pump housing 41, and the power supply which is not shown here can for example be carried out centrally at location 43. The webs 13, 14 that are configured as vanes as shown in FIG. 1, are not necessary in this embodiment of the blood pump 30.

A similarly constructed pump conduit, which is not shown here has an angle adapted to the anatomical conditions between connecting piece and the pump housing and is likewise suited for implantation at the aortic furcation to both pelvic arties for support of the blood flow in arterial occlusionay disease in the pelvic and leg area in a situation when reconstructive vascular surgical procedures alone are not sufficiently successful.

While the invention has been illustrated and described as embodied in a blood pump with impeller, it is not intended to be limited to the details shown since various modifications and structural changes may be made without departing in any way from the spirit of the present invention. The embodiments were chosen and described, in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. A blood pump having an impeller comprising at least two vascular connection devices for a tubeless connection of the pump to a blood vessel outside the heart.

2. The blood pump of claim 1, wherein the tubeless connection devices are of the type selected from the group consisting of a suture ring and a vascular prosthesis.

3. The blood pump of claim 1, wherein the impeller is housed inside a pump housing and the connection devices are disposed at the housing.

4. The blood pump of claim 1, wherein the length of the pump housing is lass than twice that of its diameter.

5. The blood pump of claim 4, wherein the length is less than 1.5 times the diameter of the housing.

6. The blood pump of claim 1, wherein the length of each of the vascular connection devices is shorter than the diameter of the pump housing.

7. The blood pump of claim 3, further comprising a motor for driving the impeller and wherein webs are provided between an outer casing of the housing and the motor.

8. The blood pump of claim 8, wherein the webs are configured as vanes.

9. The blood pump of claim 7, wherein the motor is firmly held to the outer mantel of the housing by the webs.

10. The blood pump of claim 7, wherein the webs are configured for housing metal cables or metal pins for transmission of electric current.

11. The blood pump of claim 7, wherein an area bordered by the motor and the casing, is a flow area with a diameter which is 50% of a free flow area at one end of the pump housing.

12. The blood pump of claim, 11, wherein the diameter of the free flow area is at least 80% of the free flow diameter at the one end of the pump housing.

13. The blood pump of claim 3, wherein the pump is provided with two pump housings and two motors.

14. The blood pump of claim 14, further comprising an adaptable connection device between the two pump housings.

15. The blood pump of claim 14, wherein the impellers can be driven in opposite direction.

16. The blood pump of claim 3, further comprising an auxiliary motor disposed at the housing, said auxiliary motor provided with a mass driven in opposite direction to the impeller.

17. The blood pump of claim 3, wherein the housing is provided with an attachment device for attachment of the pump to tissue.

18. The blood pump of claim 17, wherein the attachment device is configured for attachment to a rib cage.

19. A method for a tubeless vascular implant of a blood pump with an impeller comprising the steps of providing a blood pump with connection devices, preparing vascular tissue for the implant, inserting the pump into location and connecting the pump directly to the vascular tissue with a connecting device selected from the group consisting of suture rings and vascular prosthesis.

20. The method of claim 19, wherein the pump connection devices are sutured to the vascular tissue.
